(19) United States
(12) Patent Application Publication
(10) Pub. No.: US 2008/0021568 A1
(43) Pub. Date: Jan. 24, 2008

(54) Acetabular cup augment system

(75) Inventors: Peter Tulkis, Paramus, NJ (US); Ryan James Laurent, Wayne, NJ (US); David A. McQueen, Kechi, KS (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/483,313

(22) Filed: Jul. 7, 2006

(51) Int. Cl. A61F 2/34

(52) U.S. Cl. 623/22.35; 623/22.38

(57) ABSTRACT

A modular prosthetic acetabular cup for use in restorative hip replacement has an augment which can be attached to an acetabular cup outer shell to provide an acetabular cup with a cross section of a desired configuration. The augment can be attached to the acetabular cup by a coupling element having an outer dovetail portion which slidably engages a groove formed within the augment preferably open to at least a first end thereof. The inner end of the coupling element can engage screw holes of the acetabular cup. The groove of the augment further includes a second end having a gradually increasing distance from the outer surface of the shell and the inner surface of the augment on moving towards the second end of the augment.

### FIELD OF THE INVENTION

This invention relates to artificial joint implants. More particularly, this invention relates to modular, multi-component acetabular cup joint implants. Specifically, this application relates to the use of modular augments to fill bone defects in the acetabulum.

### BACKGROUND OF THE INVENTION

Prosthetic acetabular cups are well known for use in total hip arthroplasty. In such a surgery the head of the femur is replaced by a prosthetic femoral component which includes a part-spherical ball designed to engage the bearing component of a prosthetic acetabular cup.

During primary total hip arthroplasty generally an acetabular cup with a hemispherical outer surface is utilized with either bone cement or by a press-fit within a prepared acetabulum. In either case the outer shell of the prosthetic acetabular cup can include apertures for receiving bone screws or pins which aid in fixation of the outer shell within the acetabulum. Once fixed the bearing liner, usually a polyethylene insert having a part spherical inner recess adapted to receive the prosthetic head of the femoral component, is inserted within the shell.

In some cases during primary and especially during revision total hip arthroplasty the acetabulum may include a bone defect such as the presence of a void usually in the superior or superior/posterior acetabular region. Such voids or defects may be caused by superior or superior/posterior migration of a previously implanted primary acetabular prosthesis such as may be encountered during revision surgery. In those circumstances, the surgeon typically must fill the superior portion of the acetabulum with bone grafts, ream a hemispherical cavity, and insert a new acetabular cup outer shell. Not only is this time consuming and expensive but exposes the patient to additional risk since bone allografts may present potential health risks due to spread of infectious diseases. Additionally, there may be defects in the inferior acetabular which can be filled by augments.

It is desirable to use a sterilized, preferably metallic, augment which can be coupled to the outer surface of the shell in the superior or superior/posterior direction to fill such defects. Such modular acetabular cups are shown in U.S. Pat. Nos. 5,176,711, 5,370,704 and 5,326,368. These patents disclose augments which can be attached to the outer surface of the shell.

### SUMMARY OF THE INVENTION

A first aspect of the present invention is an acetabular implant. Preferably including a shell, a coupling element, and an augment. The shell preferably has an inner recessed surface for receiving a bearing component, which in turn receives a femoral head, and a part spherical outer surface. The augment has an inner surface that generally conforms to the outer surface of the shell. The coupling element preferably has an enlarged inner end which may be enlarged or threaded and an outer tapered portion. The inner end can be configured to mount to an inner surface of the shell while the tapered outer portion can be configured to extend outwardly from the outer surface of the shell. The augment may further include a groove open toward the shell forming an inner bottom surface and a plurality of inner side surfaces inside the augment. The groove can have a first end and a second end, the first end can be configured to receive the outer tapered portion of the coupling element while the second end can be configured so that movement of the coupling element towards the second end compressively engages and locks the outer tapered portion of the coupling element to the inside of the groove. Thus, the inner surface of the augment compressively engages to the outer shell surface.

The enlarged outer portion of the coupling element and the groove of the augment can have a dovetail shape. Alternatively, the outer portion and groove can have a T-shape.

The distance from the bottom surface at the first end of the groove to the inner surface of the augment adjacent the shell outer surface is less than a distance from the bottom surface at the second end of the groove to the inner surface of the augment. The distance from the bottom surface of the groove to the inner surface of the augment preferably gradually increases as the groove extends towards the groove second end.

The shell can include an aperture or a plurality of apertures extending from the inner surface of the shell to the outer surface of the shell. The enlarged inner end of the coupling element can be configured to engage a recessed surface surrounding an aperture of the inner surface of the shell. The recessed surface can be a part-spherical depression and the enlarged inner end of the coupling element can preferably have a part-spherical surface for engaging the part-spherical depression. Any of the apertures can be a threaded hole and the enlarged inner end of the coupling element can be threaded for engaging any of the apertures.

The enlarged inner end of the coupling element can be received within an aperture of the shell and can be shaped eccentrically so that it can be locked into the aperture by rotating the coupling element approximately 90 degrees.

An alternate embodiment of the acetabular implant aspect of the present invention preferably including a shell with at least one aperture in the shell, a coupling element, and an augment. The shell preferably can have an inner surface for receiving a bearing element which in turn receives a femoral head and an outer surface that generally conforms to a bottom surface of an augment. The coupling element preferably has an enlarged inner end and a tapered outer portion. The enlarged inner end can be configured to mount to the shell from the inside while the tapered outer portion can be configured to extend outwardly beyond the outer surface of the shell. The augment preferably has an inner surface generally conforming to the outer surface of the shell which typically is spherically shaped. The augment further includes an arcuate groove having an inner bottom surface and a plurality of inner side surfaces inside the augment. The groove has a first end and a second end, the first end can be configured to receive the tapered outer portion of the coupling element while the second end of the groove can be configured to cause the coupling element to be placed under tension to couple the augment to the shell outer surface. The side surfaces of the arcuate groove of the augment extends generally perpendicular to the generally hemispherical outer surface of the shell, however, in the preferred embodiment, the depth of the groove changes to develop the tension in the coupling element.

Yet another acetabular implant embodiment preferably includes a shell, at least one aperture in the shell, a coupling element, and an augment. The shell has an inner surface for receiving a bearing component which in turn receives a femoral head and part spherical outer surface. The coupling element preferably has an enlarged inner end and a tapered outer portion. The inner end can be configured to mount to the outer surface of the shell by insertion from the outside of the shell while the outer portion can be configured to extend outwardly from the outer surface of the shell. While the inner end of the coupling element is preferably enlarged it may also be threaded to engage a threaded bore in the shell. The augment preferably has an inner surface generally conforming to the shape of the outer surface of the shell. The augment may further include an arcuate groove open to the bottom and having an inner bottom surface and a plurality of inner side surfaces inside the augment. The groove has a first end and a second end, the first end is configured to receive the tapered outer portion of the coupling element while the second end of the groove is configured to cause the coupling element to be placed under tension to thereby couple the augment to the shell. This is caused by tension between the enlarged tapered portion of the coupling element and the inside of the groove. The augment preferably includes at least one coupling element extending from the bottom surface of the augment into the at least one channel of the shell.

The method includes placing the enlarged inner end of the coupling element into an aperture of the shell. A first method of assembling the augment would include pushing the coupling element through the shell from the inside, then putting the groove and the augment over the tapered outer portion of the coupling element. This is accomplished by placing the open end of the groove, which is where the bottom surface of the groove is closest to the inner surface of the augment (and then sliding the augment towards the equator of the acetabular cup shell outer surface so that the coupling element moves toward the part of the groove which has a bottom surface spaced farther away from the inner surface of the augment. If both the inner surface of the groove and the outer enlarged surface of the coupling element have matching tapers, this allows a more positive locking between the two parts. A force such as that applied by the surgeon with a mallet can be used to impact the augment driving the coupling element towards the end of the groove furthest from the inner augment surface thereby locking the two pieces together. Disassembly can occur by applying the force in the opposite direction. Essentially whether the coupling element is first inserted into the shell or first inserted into the augment is a matter of design choice.

The augment can be adapted to engage an acetabular cup to provide a modular acetabular cup device that substantially conforms to the shape of the existing cavity in the pelvis and provides a cross section of a desired configuration. The augment preferably includes an open part-spherical surface that terminates in a base section. The acetabular cup can have a part-spherical outer surface that includes a locking system which engages the augment to substantially prevent relative movement between the acetabular cup and the augment. Such a locking system may include extending ribs, anti-rotation keys, dove-tail joints, mechanical fasteners or taper members.

The outer surface of the augment can be spherical or oval shaped to enable the device of the invention to substantially conform to cavities of various configurations. The outer surface of the augment can be at least partially oval in cross section and extend up to about 90 degrees to a polar axis through the center of the augment through an arc around the rim of the cup of about 180 degrees. Optionally, a layer of bone cement may be provided between the acetabular cup and the augment.

Both the cup component and augment also may include additional stabilizers such as spikes, fins or pegs. Both the cup component and the augment further may include bone ingrowth surfaces, such as sintered beads, cast mesh, or plasma sprayed surfaces. The stabilizers and the ingrowth surfaces can be formed of cobalt-chrome alloys or titanium alloys coated with known osteo-conductive materials, such as hydroxyapatite or tri-calcium phosphate.

Coatings such as bone morphogenic proteins (BMP) can be added to the ingrowth coatings. Specifically, OP-1 brand of bone morphogenic protein sold by Stryker Corporation may be used.

The modular acetabular cup of the invention may be packaged in a kit for convenient use. The kit may include a sterile container that carries one or more augments and acetabular cups of various sizes and configurations and devices for securing other cups/augments against each other to prevent relative movement as described above. The kit also may include mechanical fasteners such as bone screws and the like. Tools for tightening these fasteners also may be included in the kit. The sterile tray containing the acetabular cup, augments, and other components is placed in an outer envelope and is sealed with a cover to establish a package, all in a manner well known in the packaging of surgical items to be brought into the sterile environment of an operating room.

It will be seen that the present invention provides a modular acetabular cup that can be fitted into bone cavities that have a variety of shapes without the need to have available multiple acetabular cups and also to reduce the sculpturing of the acetabular cavity to a specific shape prior to or during the implant procedure. Use of the modular acetabular cups of the invention thereby simplifies the implant procedure and reduces the time required to implant an acetabular cup device. The modular acetabular cups of the invention also enable development of the most appropriately shaped implant, reduces the need to carry an inventory of differing shaped acetabular cup type implants, and reduces the use of bone grafts.

These and other aspects of the present invention will be apparent from the detailed description to follow, together with the accompanying drawings.

### BRIEF DESCRIPTION OF THE DRAWINGS

There follows a detailed description of preferred embodiments of the present invention which are to be read together with the drawings therein:

FIG. 1 is an exploded view of the acetabular implant of the present invention comprising an augment, coupling element and shell;

FIG. 2 is an assembled view of the acetabular implant of FIG. 1;

FIG. 3 is a cross-sectional view of the coupling element shown in FIG. 1;

FIG. 4 is a bottom view of an assembled system according to a second embodiment of the coupling system with the augment of FIG. 1;

FIG. 5 is a cross-sectional view of the assembly of FIG. 4 along lines 5-5;

FIG. 6 is a cross-sectional view along lines 6-6 of FIG. 5;

FIG. 7 is a side view of the shell and a second augment embodiment of the present invention in a partially assembled position;

FIG. 8 is an end view of the assembled system of FIG. 7;

FIG. 9 is a bottom view of the augment of FIGS. 7 and 8;

FIG. 10 is a top view of the augment shown in FIG. 9;

FIG. 11 is a cross-sectional view along lines 11-11 of FIG. 9 showing the groove within the augment;

FIG. 12 is a cross-sectional view along lines 12-12 of FIG. 10 showing the groove varying in depth through the augment;

FIG. 13 is a bottom view of a first shell embodiment;

FIG. 14 is a cross-sectional view along lines 14-14 of FIG. 13;

FIG. 15 is an enlarged bottom view of the coupling element of the present invention;

FIG. 16 is an end view of the coupling element of FIG. 15;

FIG. 17 is a side view of the coupling element of FIG. 15;

FIG. 18 is a bottom view of an augment and shell of a second embodiment of the present invention;

FIG. 19 is a cross-sectional view of the assembly of FIG. 18 along lines 19-19;

FIG. 20 is a partial assembled view of the shell and augment of FIGS. 18 and 19;

FIG. 21 is an exploded isometric view of the augment, coupling element and shell of the embodiment of FIG. 18 prior to assembly;

FIG. 22 is an isometric view of the augment of the embodiment shown in FIG. 18;

FIG. 23 is a cross-sectional view of the augment shown in FIG. 22 including a cross-sectional view of the shell and coupling element shown in FIG. 19;

FIG. 24 is a cross-sectional view along lines 24-24 of FIG. 22;

FIG. 25 is an end view of the augment of FIG. 22;

FIG. 26 is a bottom view of the augment of FIG. 22;

FIG. 27 is a cross-sectional view of the shell of FIG. 18 showing the part spherical recess surrounding the aperture in the shell;

FIG. 28 is a bottom view of the coupling element utilized in FIG. 21;

FIG. 29 is an end view of the coupling element of FIG. 28; and

FIG. 30 is a side view of the coupling element of FIG. 28.

### DETAILED DESCRIPTION

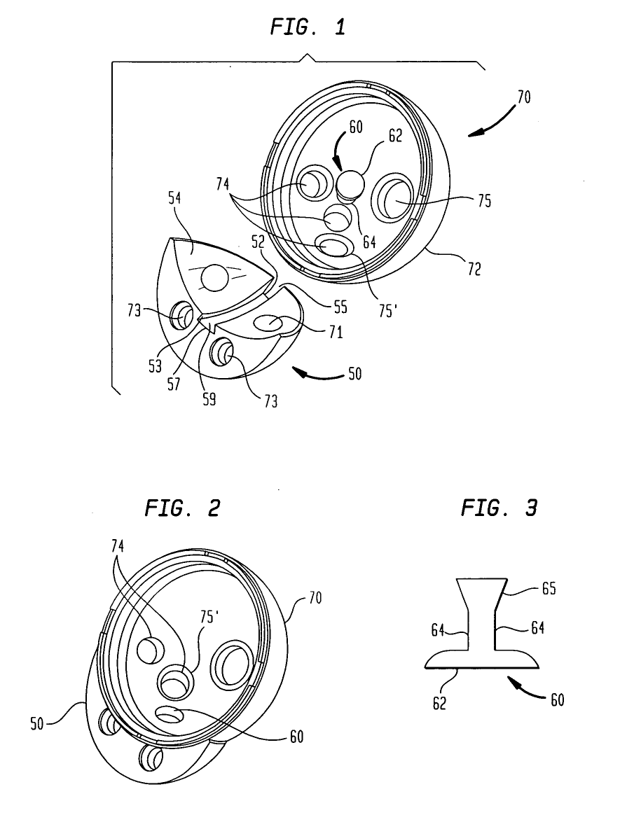

Referring to FIG. 1, there is shown an augment generally denoted as 50 which can be constructed of either a solid biocompatible metal with an externally treated or coated surface for bony attachment, or made entirely of a porous biocompatible metal for bony ingrowth. The augment 50 can be totally solid, but preferably it is bored out in strategic regions 71, so as to allow bone screws to be placed through a shell 70 into bone without obstruction. The augment could also include additional screw holes 73 for screw placement through the augment 50 and into the bone without first passing through shell 70. A polar hole 75 provided for an insertion tool attachment.

FIGS. 1-3 show a preferred embodiment of the acetabular cup augment system. FIG. 1. is an exploded view of this embodiment of the acetabular cup augment system comprising augment 50, a coupling element 60 which may be inserted into apertures 74 from the inside of shell 70. In the preferred embodiment FIGS. 1 and 3 show a tapered coupling element 60. In this embodiment the coupling element 60 can be inserted from the interior of outer shell 70 and oriented so as to be slidable in groove 52 of augment 50. For the assembly of the augment 50 to the acetabular shell 70, as seen in FIGS. 1 and 3, preferred coupling element 60 has a shell mating feature. The preferred coupling element 60 has an inner part-spherical flanged portion 62 and an outer extension portion 64. The inner portion 62 of the coupling element 60 is designed to fit into a recessed area 75′ around one of a plurality of apertures 74 of the shell 70 from the inside out. These apertures are normally used for bone screws. The extension portion 64 of coupling element 60 can fit through bores 74 and is outwardly tapered at its end 65 to interface with a tapered dovetail channel or groove 52 cut into an inner side surface 54 of the augment 50. In the embodiment of FIGS. 1-3 end 65 is inserted through holes 74 and therefore has a diameter smaller than hole 74. The channel 52 of the augment 50 has side walls 53 of a varying depth. In the preferred embodiment the depth is greater at first end 55 and less at second end 57 of channel 52. The inner facing surface 54 of the augment 50 is generally hemispherical to mate with the generally hemispherical outer surface 72 of shell 70. In the preferred embodiment augment 50 includes a pair of bores 71 and 73 for receiving typical bone screws.

To assemble the augment 50 to the shell 70, the coupling element 60 is placed from the inside of shell 70 through one of the plurality of apertures 74 of shell 70 such that outer tapered portion 65 of coupling element 60 protrudes outwardly from the generally hemispherical outer surface 72 of shell 70. The augment 50 is then placed against the outer surface 72 of the shell 70 adjacent the second end 57 of dovetail channel 52 such that the outwardly tapered portion 65 of the coupling element 60 is aligned in the groove or channel 52 of the augment 50. Once positioned, the augment 50 is translated along the outer surface 72 of the shell 70. Because the groove 52 has sidewalls 53 and a bottom surface 59 at end 55 further from the inner surface 54 of augment 50 than at end 57 the tapered portion 65 of the coupling element 60 rides deeper and deeper into the matching dovetail of groove 52. Thus, coupling element 60 is tensioned as the augment is translated. The engagement places extension 60 into greater tension as the depth of the slot increases. As force is applied in moving augment 50 away from the polar hole 75 of shell 70 by moving the end 65 of coupling element 60 deeper in groove 52, the tension eventually causes the pressure between shell 70 outer surface 72 and augment 50 inner surface 54 to build, creating a lock between the two components. As the groove 52 deepens matching side walls 64 of coupling element 60 extend from the dovetail section of the channel groove 52 to the inner surface 54 of augment 50.

In this embodiment, a plurality of coupling elements 60 can be provided having side wall portions 64 of varying length which can vary the location on surface 72 at which augment 50 locks onto the shell 70. Thus the location along groove 52 between ends 55 and 57 at which the augment locks can be easily and predictably varied. Because inner spherical surface 54 matches outer spherical surface 72 this allows variable rotation of the augment 50 about one of the plurality of apertures 72 prior to translation and locking. This relationship also allows placement of coupling element 60 in any of a plurality of apertures 74 about the shell 70.

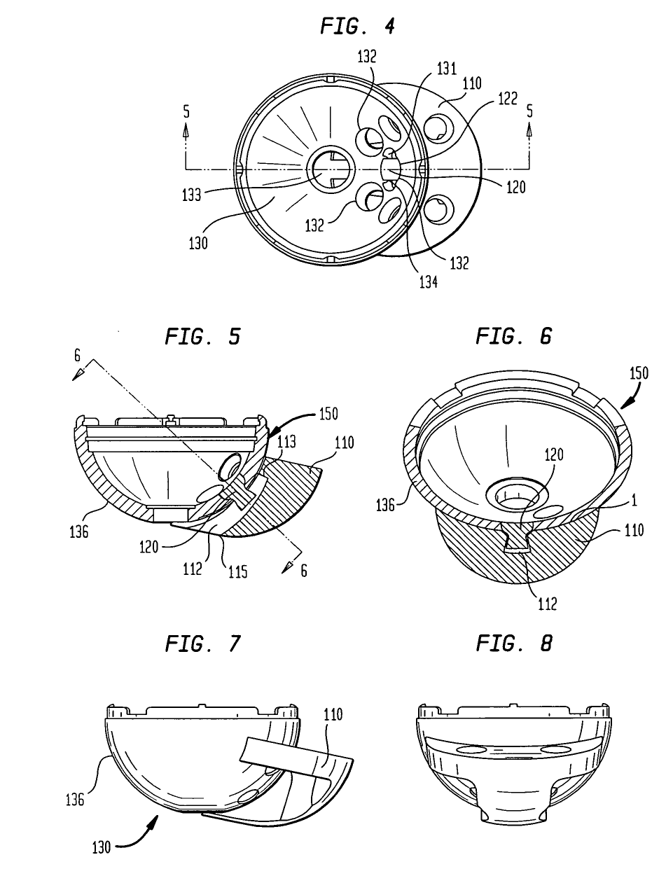

FIGS. 4-8 depict an alternative embodiment of the coupling element and augument of the acetabular cup augment system of FIGS. 1-3, including an augment 110, coupling element 120, and shell 130. This alternative assembly of the second embodiment of the invention allows assembly of the coupling element 120 into a groove 112 of the augment 110 to be performed first. Then, once contained in the augment groove 112, the coupling element 120 can be inserted from the outside (above surface 72) of shell 70 into a specialized oblong slotted screw hole 131 of the shell 130. As best seen in FIGS. 15-17 coupling element 120 includes a non-circular inner portion 122 which engages the hole 131.

FIGS. 15-17 show different views of the configuration of the coupling element 120 of the acetabular implant of FIG. 4. The coupling element 120 has an outwardly tapered end 124 to interface with the groove 112 of the augment and a flat end 122 preferably with a pair of spherical extension portions 126 to interface with at least one screw placement apertures 132 having the radial slot 134.

Hole 131 has an oblong shape which allows the inner end 122 of the coupling element to be inserted in the slotted hole 131 in a first orientation and rotated 90° to lock the coupling element 120 to shell 130. Referring to FIG. 4, the shell 130 has a plurality of bone screw placement holes or apertures 132 to receive bone screws. At least one of these plurality of apertures is a hole 131 which has a radial extension 134 for receiving non-circular end 122 of coupling element 120. The radial extensions 134 create a specialized slotted hole 131 for receiving the eccentric end 122 of coupling element 122. Referring to FIGS. 15-17 coupling element 120 is shown with elongate non-circular end 122 and dovetail end 124 for engaging groove 112. The coupling element end 122 is aligned and inserted through the ends 134 of hole 131, then turned 90 degrees to prevent disassembly.

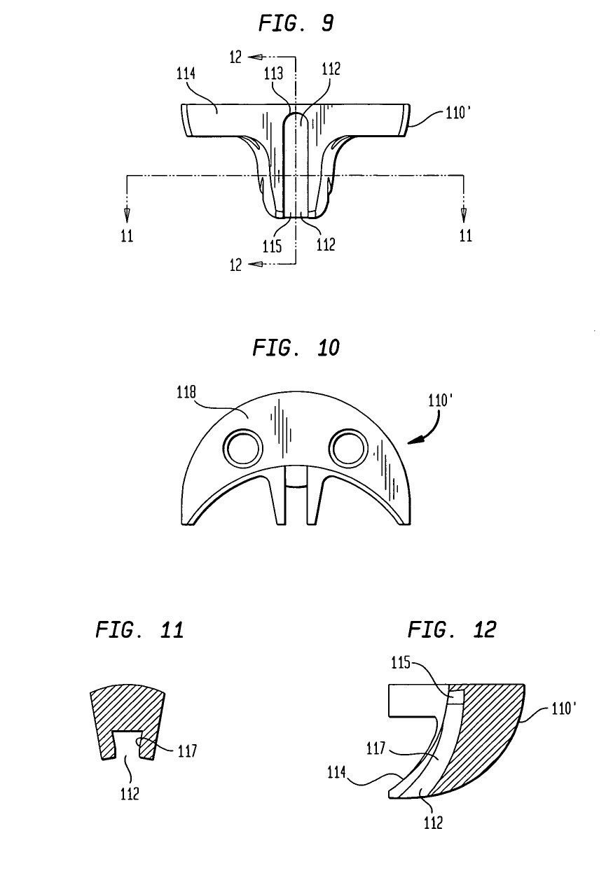

In FIGS. 4-12 an alternative augment 110′ is depicted. The augment has a bottom surface dovetail groove 112 cut into an inner surface 114 of augment 110′ for receiving the dovetail end 124 of coupling element 120. The inner surface 114 of augment 110′ is generally hemispherical and mates with the generally hemispherical outer surface 136 of shell 130. The dovetail groove 112 allows the tapered sides of dovetail end 124 of the coupling element 120 to be placed in tension and wedges the augment tightly against the acetabular shell 130. In the second embodiment, the end 124 of coupling element 120 must first be inserted into the deeper end 115 of the groove 112 of the augment 110, 110′, and then slid to end 113 and then end 122 is inserted into hole 131 of the acetabular shell 130. The augment 110, 110′ is then turned 90° and slid along shell surface 136 so that end 124 of element 120 is moved towards the deeper end of the groove 112 as described above to lock the augment in place.

FIGS. 11 and 12 are cross sectional views which depict the configuration of groove 112 of augment 110′. In FIG. 11, the groove 112 of the augment 110′ can be seen, which has a dovetail with a tapered dimension that is slightly larger than that of the tapered end 124 of the coupling element 120. Therefore, the slightly smaller tapered end 124 of the coupling element 120 can fit into the groove 112 at a deeped end 115 and slid to end 113 can be wedged tightly against inner tapered side wall surfaces 117 of the augment 110′ as the tapered end 124 of the coupling element 120 is translated toward a second deeper end 115 of the groove 112 of the augment 110. In the preferred embodiment end 115 is 0.195 inches deep and end 112 is about half that or 0.99 inches deep. The depth tapers gradually from end 112 to end 115.

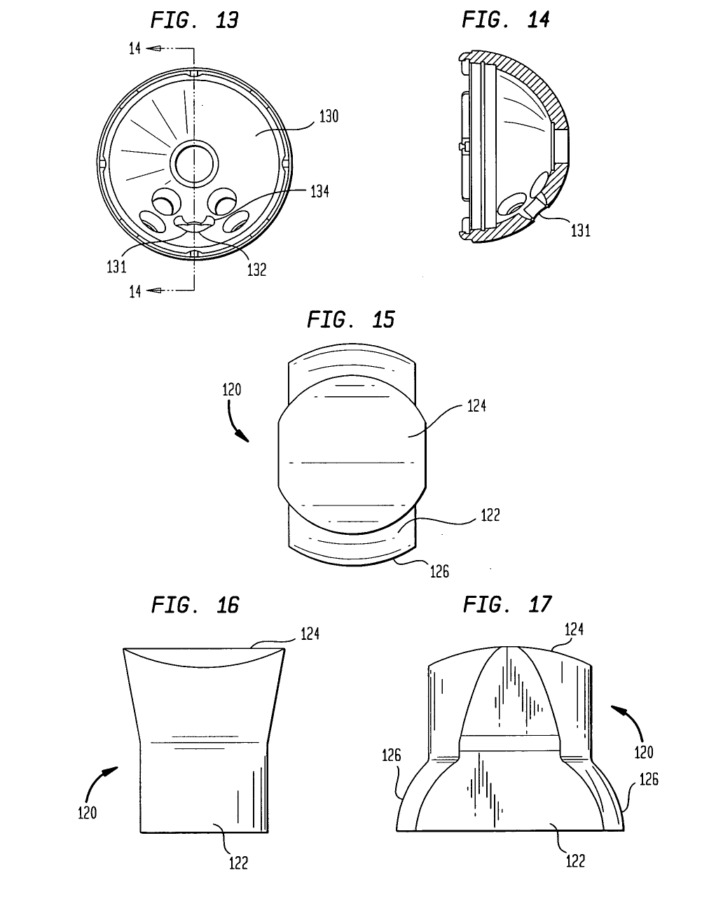

Referring to FIGS. 13 and 14 there is shown a bottom view of shell 13 with a single eccentric hole 131 for receiving end 122 of coupling element 120. FIG. 14 is a cross-sectional view through hole 132.

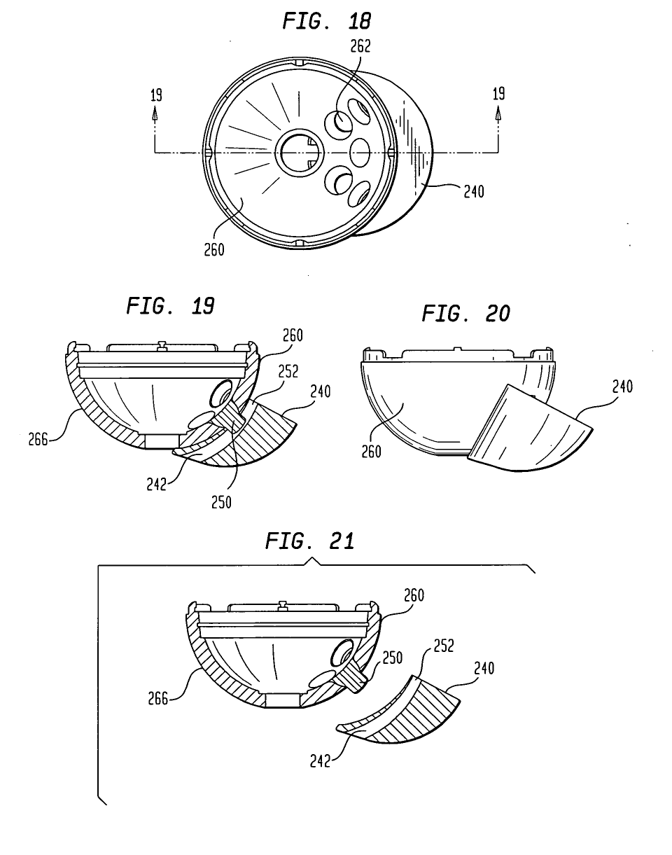

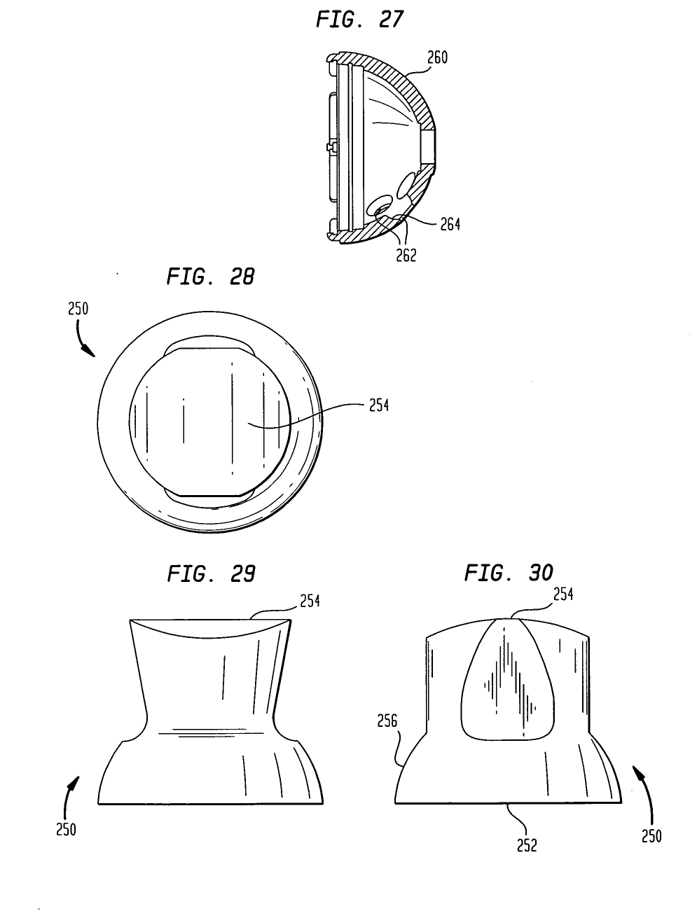

FIGS. 18-30 depict an alternate design of the acetabular cup augment system including an augment 240, coupling element 250, and shell 260. This alternative assembly of the invention allows assembly of the coupling element 250 into any of a plurality of holes 262 in the shell having part-spherical seats to receive screws having part spherical heads. FIGS. 28-30 show different views of the configuration of the coupling element 250. The coupling element 250 has a tapered end 254 to interface with the groove 242 of the augment and a flat end 252 with the part-spherical end portion 256 to interface with one of a plurality screw hole apertures 262 of the shell 260 which have a corresponding part spherical recess. As shown in FIGS. 29-30 this coupling element 250 has a part-spherical shape at its end 256. As with the other augments, end 254 is a dovetail shape.

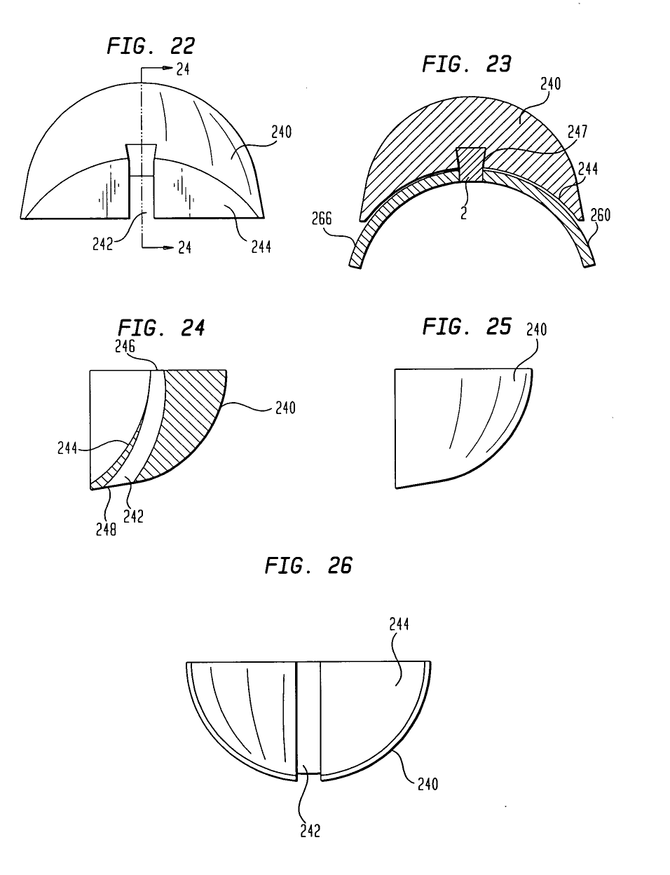

Coupling element 250 is first inserted in the shell from the inside. The augment 240 as shown in FIGS. 22 to 26 has the same tapered dovetail groove 242 cut into an inner side surface 244 of the augment for receiving the tapered end 254 of coupling element 250 as in the other embodiments. Likewise the inner side surface 244 is generally hemispherical and mates with the generally hemispherical outer surface 266 of shell 260. The tapering increasing depth of dovetail groove 242 allows the end 254 of the coupling element 250 to be wedged tightly within groove 242 as described above.

FIGS. 19 and 24 are cross sectional views which depict the configuration of augment 240. In FIG. 24, the groove 242 of the augment 240 can be seen, which has a dovetail cross-section that, as described above, is slightly larger than that of the tapered end 254 of the coupling element 250. Therefore, the slightly smaller tapered end 254 of the coupling element 250 can fit into the groove 242 at a first end 246 and can be wedged tightly against inner side surfaces 247 of the augment 240 as the tapered end 254 of the coupling element 250 is translated toward a second deeper end 248 of the groove 242 of the augment 240.

In all the embodiments described above a plurality of grooved augments of varying shapes and sizes can be provided to fill bone defects of various sizes. These augments can be provided in a kit of parts which can be placed in the operating theater. This kit would include outer shells of different sizes, coupling elements and augments to match each shell size.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acetabular implant comprising:
a shell having an inner surface and an outer surface;
a coupling element having an enlarged inner end and a tapered outer portion, the enlarged inner end being configured to the shell, the tapered outer portion being configured to extend outwardly from the outer surface of the shell; and
an augment having an inner surface generally conforming to the outer surface of the shell, the augment further including a groove, having a first and second end being configured to lockingly engage the tapered outer portion of the coupling element upon movement of the coupling element from the first to the second position in the groove.

2. The acetabular implant as set forth in claim 1, wherein the outer portion of the coupling element and the groove of the augment have a dovetail shape defining a mutual contact surface on the augment and coupling element.

3. The acetabular implant as set forth in claim 2, wherein a vertical distance from the groove contact surface at the first end of the groove to the inner surface of the augment adjacent the shell is less than a vertical distance from the contact surface at the second end of the groove to the inner surface of the augment.

4. The acetabular implant as set forth in claim 3, wherein the vertical distance from the contact surface of the groove to the inner surface of the augment gradually increases as the groove extends toward the second end.

5. The acetabular implant as set forth in claim 1, wherein the shell includes an aperture extending from the inner surface of the shell to the outer surface of the shell, the inner end of the coupling element is configured to engage a recessed surface surrounding the aperture of the inner surface of the shell.

6. The acetabular implant as set forth in claim 5, wherein the recessed surface surrounding the aperture is a part-spherical depression and the inner end of the coupling element has a part-spherical surface for engaging the part-spherical depression.

7. The acetabular implant as set forth in claim 5, wherein the shell includes a plurality of apertures.

8. The acetabular implant as set forth in claim 1, wherein the outer portion of the coupling element and the groove of the augment have a non-circular shape.

9. The acetabular implant as set forth in claim 1, wherein a non-circular inner end of the coupling element is configured to be received in a non-circular aperture in the shell and the inner end is locked into the aperture after rotating the coupling element approximately 90 degrees.

10. An acetabular implant comprising:
a shell having an inner surface and a generally hemispherical outer surface;
at least one aperture extending from the inner surface of the shell to the outer surface of the shell;
a coupling element having an enlarged inner end and a tapered outer portion, the inner end being configured to extend into the at least one aperture of the shell, the outer portion being configured to extend outwardly from the outer surface of the shell; and
an augment having an inner surface generally conforming to the outer surface of the shell, the augment further including a variable depth arcuate groove open to said inner surface of the augment, the groove having a first end having a shallower depth then a second deeper end, the second deeper end being configured to compressively engage the enlarged outer portion of the coupling element inside the groove as said coupling element is moved from said first groove end towards said second end.

11. The acetabular implant as set forth in claim 10, wherein the outer portion of the coupling element and a cross-section of the groove have a dovetail shape.

12. The acetabular implant as set forth in claim 11, wherein a vertical distance from a contact surface of said groove from the augment inner surface at the first end of the groove is less than a vertical distance from the contact surface of said groove at the second end thereof to the inner surface of the augment.

13. The acetabular implant as set forth in claim 12, wherein the vertical distance from the contact surface of the groove to the inner surface of the augment gradually increases as the groove extends toward the second end.

14. The acetabular implant as set forth in claim 10, wherein the inner surface of the shell surrounding the aperture includes a part-spherical depression.

15. The acetabular implant as set forth in claim 10, wherein the inner end of the coupling element is configured to engage a recessed surface surrounding any of the at least one aperture of the inner recessed surface of the shell.

16. The acetabular implant as set forth in claim 15, wherein the recessed surface is a part-spherical depression and the inner end of the coupling element has a part-spherical surface for engaging the part-spherical depression.

17. The acetabular implant as set forth in claim 10, wherein the enlarged inner end of the coupling element is non-circular and received by an at least one non-circular aperture of the shell, the non-circular inner end is locked into the at least one non-circular aperture by rotating the coupling element approximately 90 degrees.

18. A method of assembling an acetabular implant having a shell, comprising:
placing an inner end of a coupling element having an enlarged inner portion into an aperture of the shell such that an outer portion of the coupling element extends outwardly from an outer surface of the shell;
placing an inner surface of an augment against the outer surface of the shell such that the outer portion of the coupling element is located at a first end of a groove open to the inner surface of the augment, the groove further including a vertical distance from a coupling element contact surface of the groove at the first end of the groove to the inner surface of the augment that is less than a vertical distance from the coupling element contact surface at a second end of the groove to the inner surface of the augment; and
creating a coupling force between the outer portion of the coupling element and the augment by translating the augment with respect to the coupling element towards the second groove end.

19. The method of claim 18, wherein the shell includes a plurality of apertures.

20. The method of claim 19, wherein the step of placing the inner end of the coupling element into an aperture of the shell further includes placing the inner end of the coupling element into any of a plurality of apertures located about the outer surface of the shell.

21. A method of assembling an acetabular augment to an acetabular shell comprising;
placing an outer portion of a coupling element into a groove in the augment, the groove open to an inner surface of the augment, the groove having a first end with a contact surface for engaging the end of the coupling element spaced a first distance from the inner surface of the augment and a second end having a contact surface spaced a second distance from the inner surface of the augment, said second distance greater than said first distance;
placing the inner surface of the augment against an outer surface of an acetabular shell;
inserting a non-circular inner portion of the coupling element into a non-circular aperture in the acetabular shell, locking the inner portion of the coupling element to the acetabular shell by rotating the coupling element in said aperture; and
creating an interference fit between the outer portion of the coupling element and the second groove end by translating the coupling element towards the second groove end.
